(19) United States
(12) Patent Application Publication
(10) Pub. No.: US 2004/0157892 A1
(43) Pub. Date: Aug. 12, 2004

(54) Pesticidal fluoroethyl pyrazoles

(75) Inventors: Sheldon B. Park, Waterloo (CA); Mark A. Dekeyser, Waterloo (CA); Paul T. McDonald, Middlebury, CT (US)

Correspondence Address: Daniel Reitenbach, CROMPTON CORPORATION, 199 Benson Road, Middlebury, CT, 06749, US

(73) Assignees: CROMPTON CO./Cie; UNIROYAL CHEMICAL CO., INC.

(21) Appl. No.: 10/365,762

(22) Filed: Feb. 12, 2003

(51) Int. Cl. A01N 43/40; A01N 43/78; C07D 417/02; C 07D 4 3/02

(52) U.S. Cl. 514/341; 514/365; 548/205; 546/275.4

(57) ABSTRACT

Disclosed herein are fluoroethyl pyrazole compounds of the formula:

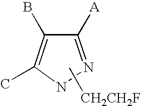

wherein A and C are independently selected from the group consisting of hydrogen, nitro, carboxyalkyl, and carboxyhaloalkyl and B is selected from the group consisting of hydrogen, nitro, arylalkynyl, 5-membered heterocycle, and 6-membered heterocycle; provided that:

A) if A and C are hydrogen, B is:

1) arylalkynyl where aryl is

a) phenyl optionally substituted with halo, haloalkyl, alkyl, alkoxy, cyano,

b) a six-membered heterocycle optionally substituted with halo, or

c) a five-membered heterocycle optionally substituted with halo;

2) a 5-membered heterocycle substituted with halo, alkyl, haloalkyl or carboxyalkyl; or

3) 6-membered heterocycle substituted with halo;

B) if B is hydrogen, A and C are independently selected from the group consisting of nitro, carboxyalkyl, and carboxyhaloalkyl; and

C) if B is nitro, A and C are independently selected from the group consisting of hydrogen, carboxyalkyl, and carboxyhaloalkyl.

These compounds are useful as insecticides and acaricides.

### BACKGROUND OF THE INVENTION

1. Field of Invention

The present invention relates to pesticidal pyrazole derivatives. More particularly, this invention relates to fluoroethyl pyrazole derivatives that exhibit activity as acaricides and insecticides. This invention also relates to acaricidal and insecticidal compositions comprising the fluoroethyl pyrazole derivatives, and to methods of controlling acarids and insects using such compositions.

2. Description of Related Art

Destruction of crops by acarids and insects presents a serious problem to agriculture and a wide variety of field crops are in need of protection from them.

U.S. Pat. No. 6,451,835 discloses fluoroethyl pyrazole compounds of the formula:

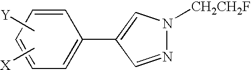

wherein X and Y are independently selected from the group consisting of hydrogen, halogen, alkyl, haloalkyl, nitro, alkylthio, alkylsulfinyl, alkylsulfonyl, and haloalkoxy. These compounds are useful as insecticides, nematocides, fungicides, and acaricides.

Baklouti and Hedhli, Journal of Fluorine Chemistry 45:255-263 (1989) (CA 112:198205), reported the preparation of certain fluoroethyl pyrazoles. They did not report biological properties of the compounds.

Japanese patent JP 59053468 1984 (CA 101:90922) refers to phenyl pyrazoles with fungicidal activity.

It is an object of the present invention to provide novel fluoroethyl pyrazole derivatives and physiologically acceptable salts thereof that are useful as acaricides and insecticides.

### SUMMARY OF THE INVENTION

The present invention relates to fluoroethyl pyrazole compounds of the formula:

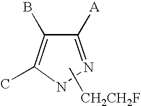

wherein A and C are independently selected from the group consisting of hydrogen, nitro, carboxyalkyl, and carboxyhaloalkyl and B is selected from the group consisting of hydrogen, nitro, arylalkynyl, 5-membered heterocycle, and 6-membered heterocycle; provided that:

A) if A and C are hydrogen, B is:

1) arylalkynyl where aryl is

a) phenyl optionally substituted with halo, haloalkyl, alkyl, alkoxy, cyano,

b) a six-membered heterocycle optionally substituted with halo, or

c) a five-membered heterocycle optionally substituted with halo;

2) a 5-membered heterocycle substituted with halo, alkyl, haloalkyl or carboxyalkyl; or

3) 6-membered heterocycle substituted with halo;

B) if B is hydrogen, A and C are independently selected from the group consisting of nitro, carboxyalkyl, and carboxyhaloalkyl; and

C) if B is nitro, A and C are independently selected from the group consisting of hydrogen, carboxyalkyl, and carboxyhaloalkyl.

The compounds of the present invention are useful as plant protecting agents for the control of acarid and insect pests. Thus, the present invention also relates to a pesticidal composition comprising: a) an effective amount of a compound of the above formula and b) a suitable carrier. The present invention further relates to a method for controlling acarids or insects that comprises applying an effective amount of the compound of the above formula to the locus to be protected.

More particularly, the present invention is directed to fluoroethyl pyrazole compounds of the formula:

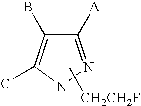

wherein A and C are independently selected from the group consisting of hydrogen, nitro, carboxyalkyl, and carboxyhaloalkyl and B is selected from the group consisting of hydrogen, nitro, arylalkynyl, 5-membered heterocycle, and 6-membered heterocycle; provided that:

A) if A and C are hydrogen, B is:

1) arylalkynyl where aryl is

a) phenyl optionally substituted with halo, haloalkyl, alkyl, alkoxy, cyano,

b) a six-membered heterocycle optionally substituted with halo, or

c) a five-membered heterocycle optionally substituted with halo;

2) a 5-membered heterocycle substituted with halo, alkyl, haloalkyl or carboxyalkyl; or

3) 6-membered heterocycle substituted with halo;

B) if B is hydrogen, A and C are independently selected from the group consisting of nitro, carboxyalkyl, and carboxyhaloalkyl; and

C) if B is nitro, A and C are independently selected from the group consisting of hydrogen, carboxyalkyl, and carboxyhaloalkyl.

In another aspect, the present invention is directed to a pesticidal composition comprising:

A) at least one fluoroethyl pyrazole compound of the formula:

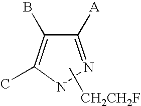

wherein A and C are independently selected from the group consisting of hydrogen, nitro, carboxyalkyl, and carboxyhaloalkyl and B is selected from the group consisting of hydrogen, nitro, arylalkynyl, 5-membered heterocycle, and 6-membered heterocycle; provided that:

1) if A and C are hydrogen, B is:

a) arylalkynyl where aryl is

i) phenyl optionally substituted with halo, haloalkyl, alkyl, alkoxy, cyano,

ii) a six-membered heterocycle optionally substituted with halo, or

iii) a five-membered heterocycle optionally substituted with halo;

b) a 5-membered heterocycle substituted with halo, alkyl, haloalkyl or carboxyalkyl; or

c) 6-membered heterocycle substituted with halo;

2) if B is hydrogen, A and C are independently selected from the group consisting of nitro, carboxyalkyl, and carboxyhaloalkyl; and

3) if B is nitro, A and C are independently selected from the group consisting of hydrogen, carboxyalkyl, and carboxyhaloalkyl; and

B) a suitable carrier.

In still another aspect, the present invention is directed to a method for controlling pests comprising applying an effective amount of at least one pyrazole compound of the formula:

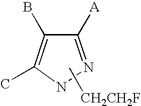

wherein A and C are independently selected from the group consisting of hydrogen, nitro, carboxyalkyl, and carboxyhaloalkyl and B is selected from the group consisting of hydrogen, nitro, arylalkynyl, 5-membered heterocycle, and 6-membered heterocycle; provided that:

A) if A and C are hydrogen, B is:

1) arylalkynyl where aryl is

a) phenyl optionally substituted with halo, haloalkyl, alkyl, alkoxy, cyano,

b) a six-membered heterocycle optionally substituted with halo, or

c) a five-membered heterocycle optionally substituted with halo;

2) a 5-membered heterocycle substituted with halo, alkyl, haloalkyl or carboxyalkyl; or

3) 6-membered heterocycle substituted with halo;

B) if B is hydrogen, A and C are independently selected from the group consisting of nitro, carboxyalkyl, and carboxyhaloalkyl; and

C) if B is nitro, A and C are independently selected from the group consisting of hydrogen, carboxyalkyl, and carboxyhaloalkyl;

to the locus to be protected.

### DESCRIPTION OF PREFERRED EMBODIMENTS

As stated above, the present invention is directed to fluoroethyl pyrazole compounds of the formula:

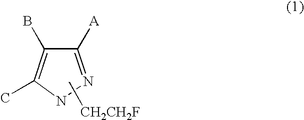

wherein A and C are independently selected from the group consisting of hydrogen, nitro, carboxyalkyl, and carboxyhaloalkyl and B is selected from the group consisting of hydrogen, nitro, arylalkynyl, 5-membered heterocycle, and 6-membered heterocycle, provided that:

A) if A and C are hydrogen, B is:

1) arylalkynyl where aryl is

a) phenyl optionally substituted with halo, haloalkyl, alkyl, alkoxy, cyano,

b) a six-membered heterocycle optionally substituted with halo, or

c) a five-membered heterocycle optionally substituted with halo;

2) a 5-membered heterocycle substituted with halo, alkyl, haloalkyl or carboxyalkyl; or

3) 6-membered heterocycle substituted with halo;

B) if B is hydrogen, A and C are independently selected from the group consisting of nitro, carboxyalkyl, and carboxyhaloalkyl; and

C) if B is nitro, A and C are independently selected from the group consisting of hydrogen, carboxyalkyl, and carboxyhaloalkyl.

Where A, B, or C comprise a substituent that is alkyl or comprises an alkyl moiety, e.g., alkoxy, carboxyalkyl, etc., it is preferred that such alkyl or alkyl moiety be an alkyl group of from 1 to 8 carbon atoms, more preferably, a lower alkyl group, e.g., one having from 1 to 5 carbon atoms.

The compounds of the instant invention can be prepared by reacting a pyrazole of formula 2 with 2-bromofluoroethane in the presence of a hydrogen halide, e.g., HBr, acceptor, such as potassium carbonate or sodium hydroxide in a solvent such as dimethylsulfoxide or acetonitrile.

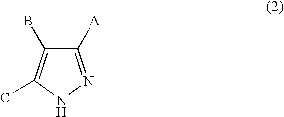

Compounds of formula 2 in which A or B is nitro and A or C is carboxyalkyl or carboxyhaloalkyl can be prepared by reacting a compound of formula 3 with an alcohol in the presence of an acid catalyst, such as sulfuric acid.

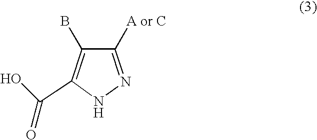

Compounds of formula 2 in which B is a heterocycle and A and C are hydrogen can be prepared by reacting a pyrazole of the formula 4 with aqueous acid, e.g., HCl. Optionally, this may be done in a suitable solvent, such as methanol or tetrahydrofuran.

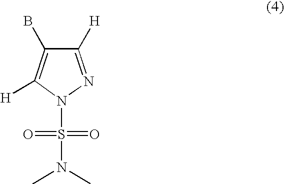

Compounds of formula 4 where A and C are hydrogen can be prepared by reacting a pyrazole of formula 5 with a heteroaryl halide, e.g., 2-bromo-5-chlorothiophene, in the presence of a suitable catalyst system, such as a mixture of palladium (II) acetate and triphenylphosphine or a mixture of palladium (II) acetate and 2-(di-t- butylphosphine)biphenyl in a suitable solvent, such as toluene or dimethylformamide.

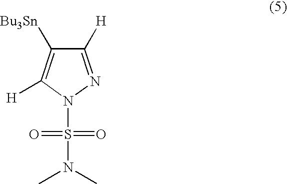

Compounds of formula 5 may be prepared by the reaction of compounds of formula 6 with alkyllithium, e.g., butyllithium, followed by reaction with tributylstannyl chloride in a suitable solvent, such as diethyl ether.

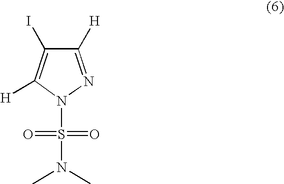

Compounds of formula (1) in which B is a arylalkynyl can be prepared by reaction of a pyrazole of formula 7 with an arylacetylene of formula 8 in the presence of a suitable base, such as potassium carbonate, and a suitable catalyst system, such as tetrakis(triphenylphosphine)palladium(0) or palladium on carbon and triphenylphosphine, and copper(I) iodide in the presence of a suitable solvent mixture, such as 1,4-dioxane/water, tetrahydrofuran/water.

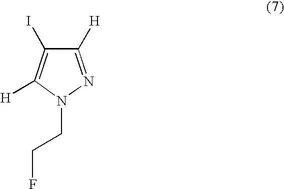

HC≡C-Aryl (8)

The present invention further relates to a pesticidal composition comprising a) an effective amount of a fluoroethyl pyrazole derivative of formula 1; and (b) a suitable carrier. Such suitable carriers may be solid or liquid in nature.

Suitable liquid carriers can comprise water, alcohols, ketones, phenols, toluene, and xylenes. In such formulations, additives conventionally employed in the art can be utilized, such as one or more surface active agents and/or inert diluents, to facilitate handling and application of the resulting insecticidal composition.

Alternatively, the compounds of this invention can be applied as a liquid or in sprays when utilized in a liquid carrier, such as a solution comprising a compatible solvent, such as acetone, benzene, toluene, or kerosene, or a dispersion comprising a suitable non-solvent medium such as water.

The compositions of this invention can alternatively comprise solid carriers taking the form of dusts, granules, wettable powders, pastes, aerosols, emulsions, emulsifiable concentrates, and water-soluble solids. For example, the compounds of this invention can be applied as dusts when admixed with or absorbed onto powdered solid carriers, such as mineral silicates, talc, pyrophyllite, and clays, together with a surface-active dispersing agent so that a wettable powder is obtained which then is applied directly to the loci to be treated. Alternatively, the powdered solid carrier containing the compound admixed therewith can be dispersed in water to form a suspension for application in such form.

Granular formulations of the compounds are preferred for field treatment and are suitable for application by broadcasting, side dressing, soil incorporation, or seed treatment, and are suitably prepared using a granular or pelletized form of carrier, such as granular clays, vermiculite, charcoal, or corn cobs. The compounds of this invention can be dissolved in a solvent and sprayed onto an inert mineral carrier, such as attapulgite granules (10-100 mesh), whereupon the solvent is evaporated. Such granular compositions can contain from 2-25% of a compound of this invention, based on carrier plus compound, preferably, 3-15%. In addition, the compounds of this invention can also be incorporated into a polymeric carrier, such as polyethylene, polypropylene, butadiene-styrene, styrene-acryonitrile resins, polyamides, poly(vinyl acetates), and the like. When encapsulated, the compounds of this invention can advantageously be released over an even longer time period, extending their effectiveness further than when used in non-encapsulated form.

Another method of applying the compounds of this invention to the loci to be treated is by aerosol treatment, for which the compounds can be dissolved in an aerosol carrier that is a liquid under pressure, but which is a gas at ordinary temperature (e.g., 20° C.) and atmospheric pressure. Aerosol formulations can also be prepared by first dissolving the compounds in a less volatile solvent and then admixing the resulting solution with a highly volatile liquid aerosol carrier.

For treatment of plants (such term including plant parts), the compounds of the invention preferably are applied in aqueous emulsions containing a surface-active dispersing agent, which can be non-ionic, cationic, or anionic. Suitable surface-active agents are well known in the art, such as those disclosed in U.S. Pat. No. 2,547,724 (columns 3 and 4). The compounds of the present invention can be mixed with such surface-active dispersing agents, with or without an organic solvent, as concentrates for the subsequent addition of water, to yield aqueous suspensions of the compounds at desired concentration levels.

In addition, the compounds can be employed with carriers which themselves are pesticidally active, such as insecticides, acaricides, fungicides, or bactericides.

It will be understood that the effective amount of a compound in a given formulation will vary depending, e.g., upon the specific pest to be combated, as well as upon the specific chemical composition and formulation of the compound being employed, the method of applying the compound/formulation, and the locus of treatment. Generally, however, the effective amount of the compounds of this invention can range from about 0.1 to about 95 percent by weight. Spray dilutions can be as low as a few parts per million, while at the opposite extreme, full strength concentrates of the compound can be usefully applied by ultra low volume techniques. When plants constitute the loci of treatment, concentration per unit area can range from between about 0.01 and to about 50 pounds per acre, with concentrations of between about 0.1 and about 10 pounds per acre preferably being employed for crops such as corn, tobacco, rice, and the like.

To combat pests, sprays of the compounds can be applied to any suitable locus, such as to the pests directly and/or to plants upon which they feed or nest. The compositions of this invention can also be applied to the soil or other medium in which the pests are present. The specific methods of application of the compounds and compositions of this invention, as well as the selection and concentration of these compounds, will vary depending upon such circumstances as crops to be protected, geographic area, climate, topography, plant tolerance, and the like.

The compounds of the invention are particularly useful as insecticides and acaricides for foliar and/or soil application. The compounds are particularly effective for controlling insects, such as corn rootworm, which live in the soil during one or more phases of their lives, by means of soil application.

The advantages and the important features of the present invention will be more apparent from the following examples.

### EXAMPLES

### Example 1

### Preparation of Isopropyl 1-(2-fluoroethyl)-3(or 5)-nitro-1H-pyrazole-5 (or 3)-carboxylate

### Compound 1

Three drops of sulfuric acid was added to a solution of 2 grams of 5-nitro-3-pyrazolecarboxylic acid in 10 mL of isopropanol. The mixture was heated to 100° C. for 16 hours and then the solvent was evaporated under reduced pressure. The remaining oil was diluted with aqueous sodium bicarbonate and extracted with 40 mL of dichloromethane. Evaporation of the dichloromethane extract produced isopropyl 5-nitro-3- pyrazolecarboxylate as a solid. To 140 mg of this intermediate was added 80 mg of potassium carbonate and 2 mL of dimethyl sulfoxide. The reaction mixture was stirred for 20 minutes before adding 75 mg of 1-bromo-2-fluoroethane. The mixture was heated to 60° C. for 2 hours. After cooling to room temperature, 30 mL of water was added and the mixture was extracted with 100 mL of diethyl ether. The product was isolated by evaporation of the solvent.

### Example 2

### Preparation of 1-dimethylsulfamoyl-4-tributylstannylpyrazole

A solution of one gram of 1-dimethylsulfamoyl-4-iodopyrazole in 10 mL of diethyl ether was cooled in a dry-ice/acetone bath and then 2.5 mL of butyllithium (1.6 M in hexane) was added over 25 minutes. The mixture was stirred for 45 minutes and then 1.1 mL of tributylstannylchloride was added. The resulting mixture was stirred cold for 30 minutes, then warmed to room temperature. The mixture was partitioned between ether and water and the organic phase was concentrated. The residue was purified by chromatography on silica eluting with 5% ethyl acetate:hexanes to give 1-dimethylsulfamoyl-4-tributylstannylpyrazole. NMR (CDCl₃) 7.85 (s, 1 H), 7/65 (s, 1 H), 2.95 (s, 6 H), 1.5-0.9 (m, 27 H).

### Example 3

### Preparation of 1-(2-fluoroethyl)-4-(5-chloro-2-thienyl)pyrazole

### Compound 6

A mixture of 3.5 grams of 1-dimethylsulfamoyl-4-tributylstannylpyrazole, 2.4 mL of 2-bromo-5-chloro-thiophene, 1.1 grams of triphenylphosphine, and 0.24 gram of palladium (II) acetate was heated at reflux for 4 hours and at 90° C. for 16 hours. Then, 1.5 mL of 2-bromo-5-chlorothiophene was added and the mixture was heated at reflux for 6 hours. One mL of 2-bromo-5-chlorothiophene was added and the mixture was heated at 90° C. for 16 hours and then heated at reflux for 7 hours. The mixture was permitted to cool and was then filtered. The filtrate was concentrated and partitioned between hexane and acetonitrile. The acetonitrile phase was concentrated and purified by chromatography on silica, eluting with methylene chloride to give crude 2-dimethylsulfamoyl-4-(5-bromo-2-thienyl)pyrazole. This material was dissolved in 10 mL of dioxane and 10 mL of 6 M hydrochloric acid and heated on a steam bath for 1 hour. The mixture was basified with 3 M sodium hydroxide and extracted into ethyl acetate. The organic solution was concentrated and purified by chromatography on silica. The residue was stirred in 10 mL of dimethyl sulfoxide with 5 grams of potassium carbonate and 2 mL of 2-bromofluoroethane for 16 hours. The mixture was partitioned between water and ethyl acetate and the organic phase was concentrated. The crude product was purified by chromatography on silica, eluting with 50% ethyl acetate methylene chloride to give 1-(2-fluoroethyl)-4-(5-chloro-2-thienyl)pyrazole.

### Example 4

### Preparation of Ethyl 2-[1-(2-fluoroethyl)-4-pyrazolyl]-4-trifluoromethyl-5-thiazolecarboxylate

### Compound 7

A mixture of 2.0 grams of 1-dimethylsulfamoyl-4-tributylstannylpyrazole, 2.5 grams of ethyl 2-chloro-4-trifluoromethylthiazole-5-carboxylate and 0.25 gram of tetrakis(triphenylphosphine)palladium(0) in 10 mL of toluene was heated at 90° C. overnight. A solution of 36 mg of palladium (II) acetate and 96 mg 2-(di-t-butylphosphine)biphenyl in 2 mL of toluene was added and the mixture was heated for a further 30 minutes and then permitted to cool. Twenty mL of hexane was added and the mixture was filtered. The solid was purified by chromatography on silica, eluting with 25% ethyl acetate: hexane. The residue was heated in a mixture of 3 mL of ethanol/7 mL of tetrahydrofuran/2 mL of 6 molar hydrochloric acid at 70° C. for 2 hours. The mixture was permitted to cool and then poured into a saturated solution of sodium bicarbonate. The mixture was extracted into ethyl acetate and concentrated. The crude product was purified by chromatography on silica, eluting with 50% ethyl acetate:methylene chloride. A mixture of the residue, 1 gram of potassium carbonate, 1 mL of 2-bromofluoroethane and 3 mL of dimethylsulfoxide was stirred at room temperature for two hours. The mixture was then partitioned between water and ethyl acetate and concentrated to provide ethyl 2-[1-(2-fluoroethyl)-4-pyrazolyl]-4-trifluoromethyl-5-carboxylate.

### Example 5

### Preparation of 1-(2-fluoroethyl)-4-(6-chloro-2-pyridyl)pyrazole

### Compound 8

A mixture 2.0 grams of 1-dimethylsulfamoyl-4-tributylstannylpyrazole, 0.64 gram of 2,6-dichloropyridine, 48 mg of palladium (II) acetate, and 128 mg of 2-(di-t-butylphosphine)biphenyl in 10 mL of toluene was heated under a nitrogen atmosphere at 80° C. for 16 hours. A premixed solution of 12 mg palladium (II) acetate and 32 mg of 2-(di-t-butylphosphine)biphenyl in 3 mL of toluene was added and the mixture was heated a further six hours and then stirred at room temperature for 16 hours. Another 3 mL of a toluene solution of 12 mg palladium (II) acetate and 32 mg of 2-(di-t-butylphosphine)biphenyl was added and the mixture was heated at 80° C. for 4 hours, permitted to cool, and filtered. The filtrate was concentrated and purified by chromatography on silica, eluting with methylene chloride. A quantity of 0.37 gram of the residue was heated on a steam bath for 15 minutes in 2 mL of tetrahydrofuran/1 mL of 6 M HCL and then permitted to cool. It was then basified with saturated sodium bicarbonate. The mixture was partitioned between water and ethyl acetate and concentrated. A quantity of 0.13 gram of the residue was stirred in 4 mL of dimethylsulfoxide and 0.5 mL of 2-bromofluoroethane with 0.4 gram of potassium carbonate for 4 hours. The mixture was partitioned between water and ethyl acetate. The organic phase was concentrated to give 1-(2-fluoroethyl)-4-(6-chloro-2-pyridyl)pyrazole.

### Example 6

### Preparation of 1-(2-fluoroethyl)-4-[(4-chlorophenyl)ethynyl]pyrazole

### Compound 9

A mixture of 0.5 gram of 1-(2-fluoroethethyl)-4-iodopyrazole, .011 gram of 10% palladium on carbon, 40 mg of copper (I) iodide, and 0.72 gram of potassium carbonate in 5 mL of 1,4-dioxane/2 mL of water was stirred at room temperature for 30 minutes. Then, 0.72 gram of 4-chloroethynylbenzene was added and the mixture was heated at 80° C. for 16 hours. The mixture was cooled and filtered through Celite. The filtrate was partitioned between water and ethyl acetate and the organic phase was concentrated. The residue was purified by chromatography on silica, eluting with 25% ethyl acetate:hexane to give 1-(2-fluoroethyl)-4-[(4-chlorophenyl)ethynyl]pyrazole.

### Example 7

### Preparation of 1-(2-fluoroethyl)-4-[(3-pyridylethynyl)pyrazole

### Compound 10

A mixture of 0.5 gram of 1-(2-fluoroethethyl)-4-iodopyrazole, 0.011 gram of 10% palladium on carbon, 40 mg of copper (I) iodide, and 0.72 gram of potassium carbonate in 5 mL of 1,4-dioxane/2 mL of water was stirred at room temperature for 30 minutes. Then, 0.54 gram of 3-ethynylpyridine was added and the mixture was heated at 80° C. for 16 hours. The mixture was cooled and filtered through Celite. The filtrate was partitioned between water and ethyl acetate and the organic phase was concentrated. The residue was purified by chromatography on silica, eluting with 25% ethyl acetate:hexane to give 1-(2-fluoroethyl)-4-[(3 -pyridylethynyl)pyrazole.

TABLE 1

| Compound No. | A | B | C | NMR Data(CDCl₃) |
|---|---|---|---|---|
| 1 | NO₂ | H | CO₂iPr | 1.38(d, 6H) |
| | | | | 4.73-5.28(m, 5H) |
| | | | | 7.39(s, 1H) |
| 2 | NO₂ | H | CO₂CH₂CH₂F | 4.56-5.06(m, 8H) |
| | | | | 7.49(s, 1H) |
| 3 | H | NO₂ | CO₂iPr | 1.39(d, 6H) |
| | | | | 4.44-4.90(m, 4H) |
| | | | | 5.32(m, 1H) |
| | | | | 8.26(s, 1H) |
| 4 | H | NO₂ | CO₂Pr | 1.01(t, 3H) |
| | | | | 1.77(m, 2H) |
| | | | | 4.32-4.90(m, 6H) |
| | | | | 8.27(s, 1H) |
| 5 | NO₂ | H | CO₂Pr | 1.04(t, 3H) |
| | | | | 1.82(m, 2H) |
| | | | | 4.30(t, 2H) |
| | | | | 4.74-5.09(m, 4H) |
| | | | | 7.41(s, 1H) |
| 6 | H | 5-chloro-2- | H | 7.67(s, 1H), 7.62(s, 1H), |
| | | thienyl | | 6.85(m, 2H), 4.88 |
| | | | | (t, 1H), 4.72(m, 1H), |
| | | | | 4.48(t, 1H), 4.41(t, 1H) |
| 7 | H | ethyl 4- | H | 8.11(s, 1H), 8.01(s, 1H), |
| | | trifluoromethyl- | | 4.91(t, 1H), 4.75(t, |
| | | 2-pyrazolyl-5- | | 1H), 4.54(t, 1H), 4.45 |
| | | thiazolecarboxylate | | (t, 1H), 4.42(q, 2H), |
| | | | | 1.41(t, 3H) |
| 8 | H | 6-chloro-2- | H | 8.09(s, 1H), 8.02(s, 1H), |
| | | pyridyl | | 7.63(t, 1H), 7.39(t, |
| | | | | 1H), 7.15(d, 1H), 4.90 |
| | | | | (t, 1H), 4.74(t, 1H), |
| | | | | 4.52(t, 1H), 4.43(t, 1H) |
| 9 | H | 4- | H | 7.71(s, 1H), 7.70(s, 1H), |
| | | chlorophenylethynyl | | 7.42(m, 2H), 7.32 |
| | | | | (m, 2H), 4.87(t, 1H), |
| | | | | 4.71(t, 1H), 4.48(t, 1H), |
| | | | | 4.39(t, 1H) |
| 10 | H | 3- | H | 8.73(d, 1H), 7.75(m, 3H), |
| | | pyridylethynyl | | 7.29(m, 1H), 4.86 |
| | | | | (t, 1H), 4.71(t, 1H), |
| | | | | 4.49(t, 1H), 4.40(t, 1H) |

### Example 8

### Pesticide Screening

For each compound tested, a stock solution of 3000 ppm was prepared by diluting 0.008 gram of each compound into 2.67 mL of acetone and further diluting with water to make the final concentrations used for the various screens, as follows:

In the mite contact (MIC) bioassay, approximately 20 motile mites confined on a cowpea leaf were sprayed with a 300 ppm concentration and evaluated five days later.

In the mite ovicide (MIOV) bioassay, approximately 30 mite eggs on cowpea foliage were sprayed with a 100 ppm solution and egg hatch was evaluated nine days later.

In the rice planthopper systemic (RPHS) bioassay, roots of rice seedlings were immersed in a 30 ppm solution and about 10 hoppers were exposed to the foliage. Hoppers were evaluated one day later.

In the diamondback moth (DBM) bioassay, a single larva was exposed to a diet with a surface that had been coated with a 100 ppm solution, and evaluated five days later.

In the southern corn rootworm (SCR) bioassay, a single larva was exposed to a diet with a surface that had been coated with a 100 ppm solution, and evaluated five days later.

Since only a single replicate was used for the DBM and SCR screens, confirmation of mortality in an additional evaluation with three larvae at the 100 ppm rate was required to substantiate the initial observation of activity. Data for all screens that showed control of 50% or greater and gave confirmation (where required) are shown in the accompanying Table 2.

TABLE 2
Pesticidal Activity

| | TEST | | | | |
|---|---|---|---|---|---|
| | MIC | MIOV | RPHS | DBM | SCR |
| | Genus Species Stage Rate Method | | | | |
| | | | | | Diabrotica |
| | Tetranychus | Tetranychus | Sogatodes | Plutella | undecimpunctata |
| | urticae | urticae Eggs | orizicola | xylostella | Larva |
| | Motiles 300 ppm | 100 ppm | Nym & Ad | Larva 100 ppm | 100 ppm |
| Compound | contact | contact on | 30 ppm | residual | residual on |
| Number | on bean leaf | bean leaf | systemic rice | on diet | diet |
| 1 | 100 | | | 100 | |
| 2 | 100 | 100 | | 100 | |
| 3 | 100 | | | 100 | |
| 4 | | | 59 | | |
| 5 | 100 | | | | 100 |
| 6 | 100 | 100 | | 100 | |
| 7 | 100 | 100 | | | |
| 8 | 100 | 100 | | | |
| 9 | 100 | 100 | | | |
| 10 | 100 | 100 | | | |

In view of the many changes and modifications that can be made without departing from principles underlying the invention, reference should be made to the appended claims for an understanding of the scope of the protection to be afforded the invention.

What is claimed is:

1. A fluoroethyl pyrazole compound of the formula: 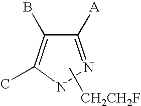
wherein A and C are independently selected from the group consisting of hydrogen, nitro, carboxyalkyl, and carboxyhaloalkyl and B is selected from the group consisting of hydrogen, nitro, arylalkynyl, 5-membered heterocycle, and 6-membered heterocycle; provided that:
A) if A and C are hydrogen, B is:
1) arylalkynyl where aryl is
a) phenyl optionally substituted with halo, haloalkyl, alkyl, alkoxy, cyano,
b) a six-membered heterocycle optionally substituted with halo, or
c) a five-membered heterocycle optionally substituted with halo;
2) a 5-membered heterocycle substituted with halo, alkyl, haloalkyl or carboxyalkyl; or
3) 6-membered heterocycle substituted with halo;
B) if B is hydrogen, A and C are independently selected from the group consisting of nitro, carboxyalkyl, and carboxyhaloalkyl; and
C) if B is nitro, A and C are independently selected from the group consisting of hydrogen, carboxyalkyl, and carboxyhaloalkyl.

2. The compound of claim 1 wherein A is NO₂, B is H, and C is CO₂iPr.

3. The compound of claim 1 wherein A is NO₂, B is H, and C is CO₂CH₂CH₂F.

4. The compound of claim 1 wherein A is H, B is NO₂, and C is CO₂iPr.

5. The compound of claim 1 wherein A is H, B is NO₂, and C is CO₂Pr.

6. The compound of claim 1 wherein A is NO₂, B is H, and C is CO₂Pr.

7. The compound of claim 1 wherein A is H, B is 5-chloro-2-thienyl, and C is H.

8. The compound of claim 1 wherein A is H, B is ethyl 4-trifluoromethyl-2-pyrazolyl-5-5-thiazolecarboxylate, and C is H.

9. The compound of claim 1 wherein A is H, B is 6-chloro-2-pyridyl, and C is H.

10. The compound of claim 1 wherein A is H, B is 4-chlorophenylethynyl, and C is H.

11. The compound of claim 1 wherein A is H, B is 3-pyridylethynyl, and C is H.

12. A pesticidal composition comprising:
A) at least one fluoroethyl pyrazole compound of the formula: 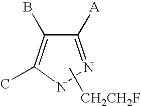
wherein A and C are independently selected from the group consisting of hydrogen, nitro, carboxyalkyl, and carboxyhaloalkyl and B is selected from the group consisting of hydrogen, nitro, arylalkynyl, 5-membered heterocycle, and 6-membered heterocycle; provided that:
1) if A and C are hydrogen, B is:
a) arylalkynyl where aryl is
i) phenyl optionally substituted with halo, haloalkyl, alkyl, alkoxy, cyano,
ii) a six-membered heterocycle optionally substituted with halo, or
iii) a five-membered heterocycle optionally substituted with halo;
b) a 5-membered heterocycle substituted with halo, alkyl, haloalkyl or carboxyalkyl; or
c) 6-membered heterocycle substituted with halo;
2) if B is hydrogen, A and C are independently selected from the group consisting of nitro, carboxyalkyl, and carboxyhaloalkyl; and
3) if B is nitro, A and C are independently selected from the group consisting of hydrogen, carboxyalkyl, and carboxyhaloalkyl; and
B) a suitable carrier.

13. The pesticidal composition of claim 12 wherein A is NO₂, B is H, and C is CO₂iPr.

14. The pesticidal composition of claim 12 wherein A is NO₂, B is H, and C is CO₂CH₂CH₂F.

15. The pesticidal composition of claim 12 wherein A is H, B is NO₂, and C is CO₂iPr.

16. The pesticidal composition of claim 12 wherein A is H, B is NO₂, and C is CO₂Pr.

17. The pesticidal composition of claim 12 wherein A is NO₂, B is H, and C is CO₂Pr.

18. The pesticidal composition of claim 12 wherein A is H, B is 5-chloro-2-thienyl, and C is H.

19. The pesticidal composition of claim 12 wherein A is H, B is ethyl 4-trifluoromethyl-2-pyrazolyl-5-5-thiazolecarboxylate, and C is H.

20. The pesticidal composition of claim 12 wherein A is H, B is 6-chloro-2-pyridyl, and C is H.

21. The pesticidal composition of claim 12 wherein A is H, B is 4-chlorophenylethynyl, and C is H.

22. The pesticidal composition of claim 12 wherein A is H, B is 3-pyridylethynyl, and C is H.

23. A method for controlling pests comprising applying an effective amount of at least one pyrazole compound of the formula: 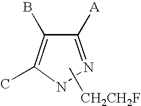
wherein A and C are independently selected from the group consisting of hydrogen, nitro, carboxyalkyl, and carboxyhaloalkyl and B is selected from the group consisting of hydrogen, nitro, arylalkynyl, 5-membered heterocycle, and 6-membered heterocycle; provided that:
A) if A and C are hydrogen, B is:
1) arylalkynyl where aryl is
a) phenyl optionally substituted with halo, haloalkyl, alkyl, alkoxy, cyano,
b) a six-membered heterocycle optionally substituted with halo, or
c) a five-membered heterocycle optionally substituted with halo;
2) a 5-membered heterocycle substituted with halo, alkyl, haloalkyl or carboxyalkyl; or
3) 6-membered heterocycle substituted with halo;
B) if B is hydrogen, A and C are independently selected from the group consisting of nitro, carboxyalkyl, and carboxyhaloalkyl; and
C) if B is nitro, A and C are independently selected from the group consisting of hydrogen, carboxyalkyl, and carboxyhaloalkyl;
to the locus to be protected.

24. The method of claim 23 wherein A is NO₂, B is H, and C is CO₂iPr.

25. The method of claim 23 wherein A is NO₂, B is H, and C is CO₂CH₂CH₂F.

26. The method of claim 23 wherein A is H, B is NO₂, and C is CO₂iPr.

27. The method of claim 23 wherein A is H, B is NO₂, and C is CO₂Pr.

28. The method of claim 23 wherein A is NO₂, B is H, and C is CO₂Pr.

29. The method of claim 23 wherein A is H, B is 5-chloro-2-thienyl, and C is H.

30. The method of claim 23 wherein A is H, B is ethyl 4-trifluoromethyl-2-pyrazolyl-5-5-thiazolecarboxylate, and C is H.

31. The method of claim 23 wherein A is H, B is 6-chloro-2-pyridyl, and C is H.

32. The method of claim 23 wherein A is H, B is 4-chlorophenylethynyl, and C is H.

33. The method of claim 23 wherein A is H, B is 3-pyridylethynyl, and C is H.

34. The method of claim 23 wherein the pest is an insect.

35. The method of claim 23 wherein the pest is an acarid.
